(19) United States
(12) Patent Application Publication
(10) Pub. No.: US 2016/0120459 A1
(43) Pub. Date: May 5, 2016

(54) Wound Acknowledgement Interface

(71) Applicant: Flawless Service Corporation, Chicago, IL (US)

(72) Inventor: Elton Xavier Tinsley, Lynwood, IL (US)

(21) Appl. No.: 14/926,677

(22) Filed: Oct. 29, 2015

Related U.S. Application Data
(60) Provisional application No. 62/072,824, filed on Oct. 30, 2014.

(51) Int. Cl. A61B 5/00; A61B 5/107; A61B 5/01

(52) U.S. Cl. CPC A61B 5/445; A61B 5/0013; A61B 5/0022; A61B 5/0077; A61B 5/7425; A61B 5/743; A61B 5/01; A61B 5/1072; A61B 5/1079; A61B 2560/0223; A61B 2560/0425; A61B 2560/0443; A61B 2576/00

(57) ABSTRACT

Systems, tools, and methods for providing wound data and/or information to a remote location for clinical assessment and acknowledgement are disclosed.

### CROSS REFERENCE TO RELATED APPLICATIONS

This application claims the benefit of U.S. application Ser. No. 62/072,824, filed Oct. 30, 2014, which is incorporated herein by reference.

### BACKGROUND

Reducing wound formation and promoting skin integrity for nursing home residents and/or other long term care facility residents affects quality of life. Governmental and national health associations have focused on skin care. For example, the Centers for Disease Control and Prevention (CDC) recently published “Pressure Ulcers Among Nursing Home Residents: United States, 2004,” in which it reported that 159,000 current U.S. nursing home residents (11%) had pressure ulcers. More recently, the CDC released the “National Hospital Medical Care Survey: 2008 Emergency Department Summary Tables,” which revealed that there are 2,504,000 emergency room visits by nursing home residents annually, and emergency rooms perform 1,862,000 wound debridements annually in the U.S.

Therefore, a large percentage of emergency room visits by nursing home residents may be related to the treatment of pressure ulcers and the like within nursing homes. Relatedly, the National Institutes of Health U.S. National Library of Medicine recently published “Human skin wounds: a major and snowballing threat to public health and the economy” in which it is estimated that the annual cost of pressure wounds alone is $8.5 billion dollars.

The Design for Nursing Home Compare Five-Star Quality Rating System, published by the Centers for Medicare-Medicaid Services (CMS) in July 2010, provides a quantitative measure of performance of nursing homes that participate in Medicare or Medicaid. It specifically rates nursing homes on the percent of residents with pressure ulcers. Coincidentally, 50% of patients with a pressure ulcer in the hospital are sent to a nursing home. That is 3 times the rate of any other reason for nursing home placement. Therefore, there is a strong incentive for nursing homes to provide vigilant care for residents who already have or are at an increased risk for acquiring pressure ulcers.

Unfortunately, residents are not necessarily checked into nursing homes when wound care specialists are readily available and onsite. This is particularly true at night and on holidays and weekends. Resident intake and skin integrity assessment is, therefore, frequently performed by staff members who are not specialists in wound care. According to CMS only 30% of U.S. nursing homes provide “specialized wound care.”

Therefore, tools are needed to assist in the identification and treatment of wounds, such as, for example, tools that would enable clinically relevant wound information to be made available to physicians remotely to permit informed clinical assessment. In addition, such tools may also facilitate family member informed consent for treatment of residents in nursing care facilities and the like. In some cases, a single tool shared by the staff, the treating physician, consultants, specialists and the families could, by way of example, help with one or more of facilitating communication, expediting specialized wound treatment, reducing complaints, speeding healing and improving quality of life for the resident.

### SUMMARY

In a first aspect, a system for providing wound data to a remote location includes an instrument adapted to capture an image of a wound area on a subject, a processing unit associated with the instrument adapted to store, process, and/or transfer the image to a remote location, and an interface adapted to receive the image and for displaying the image. The image provides information on the interface to enable a user to clinically assess the wound area on the subject based on wound volume appearance, wound area size, wound area condition, wound area topography, and wound type.

In a second aspect, a method of providing wound data to a remote location includes capturing an image of a wound area on a subject. The image provides information on an interface to enable a user to clinically assess the wound area on the subject based on wound area appearance, wound area size, and at least one of wound area topography, wound area condition and wound type. The method includes storing the image in a processing unit that allows an electronic signature acknowledging that the wound has been identified during assessment, documented by photography and shared with key persons including the treating physician, family or guardian. The responsible party present at the time of intake further signs requesting and providing consent for treatment of the identified wound(s). The method further allows transferring the image to a remote location, displaying the image on the interface, and recording a signed acknowledgment of the user based on clinical parameters evident on photography.

In a third aspect, a system for capturing an image of a wound area of a subject includes an instrument adapted to capture image data of a wound area of a subject, a wand attached to the instrument, wherein the wand has a length, and a wand tip adapted to be placed against at least one of a surface of the subject's skin adjacent the wound area and a surface of the wound area. In one embodiment, the instrument comprises a camera. In another embodiment, the wand is removably secured to the camera. In a further embodiment, the wand tip includes an analgesic compound or other medicament. In a particular embodiment, the wand and wand tip are integral. In another particular embodiment, the wand tip is removable from the wand. In a further embodiment, the wand includes a single point contact device adapted to enable a user to manipulate the relative position of the instrument with respect to the wound area in a repeatable manner without moving the wand tip. In one embodiment, the system may be encompassed within a kit. The kit may include a resealable package having a plurality of removable, disposable sterile wand tips. The kit may also include instructions for capturing image data.

In a fourth aspect, a system for providing wound information to a remote location includes an instrument adapted to capture image data of a wound area of a subject, a processing unit associated with the instrument adapted to store, process, and/or transfer the image data to a remote location, and an interface adapted to receive the image data and display an image comprising the image data. The image provides information on the interface to enable a user to clinically assess the wound area of the subject based on wound area appearance, wound area size, wound area condition, wound area topography, and wound type. In one embodiment, the image includes image data having two or more of wound area appearance, wound area size, wound area temperature, and wound area topography. In another embodiment, the image provided on the interface includes a scale along X and Y axes of the image. In a further embodiment, the system may include an image standard for distance calibration of the instrument. In another embodiment, the processing unit is adapted to calculate wound area size of the image based on the distance calibration of the instrument.

In a fifth aspect, a method of providing wound information to a remote location includes capturing an image of a wound area of a subject using an instrument, wherein the image provides information on a remote interface to enable a remote user to clinically assess the wound area of the subject based on at least wound area appearance, wound area size, wound area condition, wound area topography, and wound type, storing the image in a processing unit, transferring the image to a remote location, displaying the image on the remote interface, and recording an acknowledgment of the remote user based on the wound appearance. The acknowledgment is transferred to the instrument and stored in the processing unit in association with the image. In one embodiment, the image includes image data having two or more of wound area appearance, wound area size, wound area temperature, and wound area topography. In another embodiment, the image provided on the interface includes a scale along X and Y axes of the image. In a further embodiment, the instrument includes a removable wand for establishing a distance calibration of the instrument based on the length of the wand. In another embodiment, the processing unit is adapted to calculate wound area size of the image based on the distance calibration of the instrument.

### BRIEF DESCRIPTION OF THE FIGURES

FIG. 1 depicts a system for providing wound data to a remote location according to one embodiment;

FIG. 2 depicts an interface according to one embodiment;

FIG. 3 depicts a system for providing wound data to a remote location according to another embodiment; and

FIG. 4 depicts a system for providing wound data to a remote location according to a further embodiment.

### DESCRIPTION

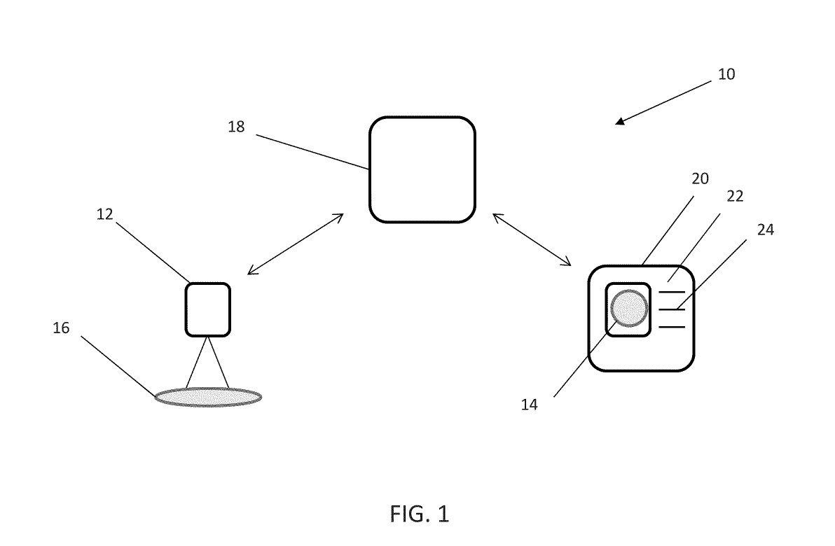

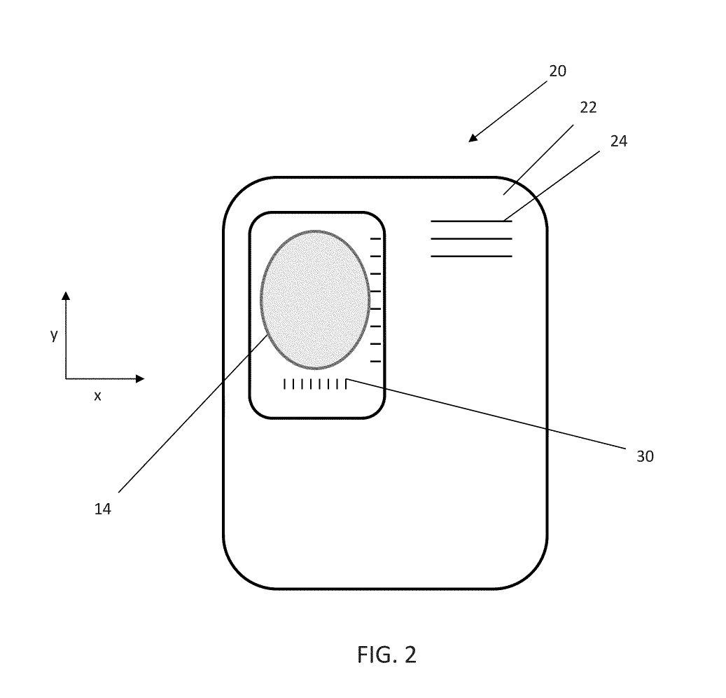

The present invention is based, at least in part, on the need for better tools for nursing homes to improve nursing home resident wound care. As seen in FIGS. 1-4, a system 10 that improves nursing home resident care by providing wound data (information) to a remote location may include an instrument 12 adapted to capture resident information, such as an image 14 of a wound area 16 of the resident, and may also be used to record or accept (e.g., by remote data transfer) resident information, such as name, medical history, family medical history, family contact information, and/or information identifying persons with power of attorney for the resident, and the like. The system 10 further includes a processing unit 18 for processing, storing, and/or relaying the image 14 and related information to a remote location. The system 10 further includes an interface 20 that is adapted to receive the resident's information, such as an image 14, from the instrument 12 at a remote location from the instrument. Direction of information flow is indicated by the arrows, which data transfer may be directly or indirectly (via internet or intervening devices) wireless or wired between electronic devices. When the interface 20 is not electronic, such as when it is a document, the data transfer is by means of a printer and the reverse direction via a scanner, printer, camera, and the like.

As used herein, a remote location may be at a location not attached to the instrument 12, such as in the same room or in a different city and the like. In one embodiment, the interface 20 may include a graphical user interface 22, which may be a touch screen and the like, upon which the image 14 and additional information 24 in text or other form may be displayed. The interface 20 is adapted to enable a user to input information, such as a wound diagnosis, wound type, a wound treatment regimen, a wound acknowledgment (e.g., recognition that a wound is present at intake by a family member or equivalent person), other instructions, and the like into the resident's file, which can be relayed to the instrument 12 or another device for action by the nursing home staff. In this way, the system 10 immediately extends the care network available to contribute to the care of a resident beyond the immediate nursing home staff and facilitates earlier documentation of and targeted intervention for a resident's wounds including wound specialists. All methods of data input are envisioned herein for the instrument 12 and the interface 20, such as, a stylus, a keyboard, a microphone, and the like.

Examples of instruments 12 with integral processing units 14 may include singly or in combination, hand held or desktop devices, such as digital cameras, “smartphones,” digital tablets, or related devices with image data capturing capability as described herein elsewhere, electronic data storage capacity, image data processing capacity, and the ability to digitally connect and receive and send information with another device either through a cable or wirelessly. As seen in FIG. 1, the processing unit 18 may be a separate device from the instrument 12, and may be chosen from stand alone computers, databases, servers, and a cloud-based storage system with data processing functionality, combinations thereof, and the like.

The interface 20 may be any device that enables the display of the information captured by the instrument 12. In one embodiment, the interface 20 may be electronic and include a graphical user interface or touch screen that enables viewing and recording of information. Examples of contemplated interfaces 20 include “smartphones,” digital tablets, personal computers, or related devices with image displaying capability, data processing capability, electronic data storage capacity, and the ability to digitally connect, store, and share information with another device either through a cable or wirelessly. In one embodiment, the instrument 12 and the interface 20 may be two of the same device, each with identical features such that each may be used as either the instrument or the interface in the system.

In one embodiment, the interface 20 may be a printed document that includes one or more of a picture, a 3D image, a hologram, a heat map indicating differences in skin surface temperature, a histogram, a graph, and the like in addition to information identifying the subject, the location of the nursing home, and the like. In this embodiment, the interface 20 may be designed for a resident's family member to receive for the purpose of acknowledging the presence of a wound on the resident. During intake, the family member may be required to acknowledge the pre-existence of a wound on the resident via signature or similar manner on the interface 20. In this way, the interface 20 may serve as a risk mitigation tool that helps to protect a nursing home from legal claims by the resident's family member for previously existing wounds (or conversely, a resident from negligence by a nursing home for new or neglected wounds).

The image information captured by the instrument 12 may provide information via the interface 20 to enable a user, such as a health care worker, to clinically assess the wound area 16 of the resident. Previously, the ability of a health care provider to clinically assess a wound area of a subject, such as a pressure wound, traumatic wound, diabetic wound, arterial wound, venous wound, burn wound, cancer wound, surgical wound, and the like, based on an image provided remotely has been limited. However, the instruments 12 of the present disclosure may record multiple types of image data in a single image or series of images including basic wound area appearance (in black and white and/or color), wound area size, wound area temperature, wound area condition (based, for example, on the presence or absence of necrotic tissue, moisture, purulent drainage, granulation tissue, debris, slough), and/or wound area topography (three dimensional images).

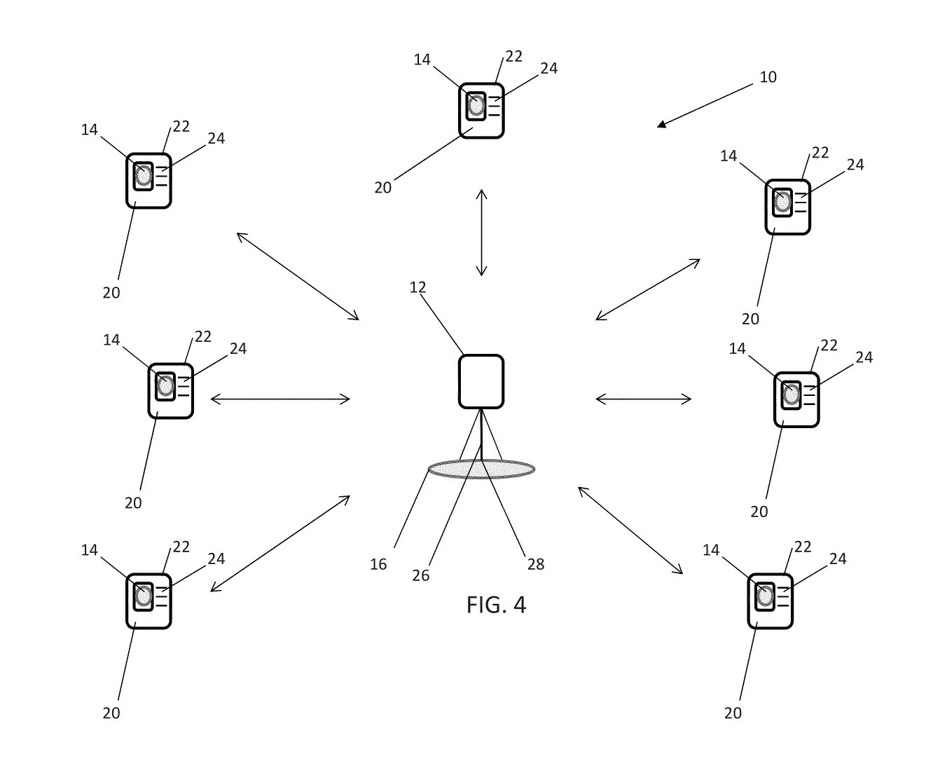

Wound area size may be accurately recorded by image capture by pre-calibrating the instrument 12 prior to first use. Calibration of the instrument 12 may be achieved by capturing an image of a standard with a known size at a known distance (calibration distance) from the instrument and assigning a size value to that image (a standard image). The size of subsequent wound area images taken at the calibration distance by the calibrated instrument 12 may be calculated automatically by software within the processing unit 18 based on the standard previously recorded. Alternatively, as seen in FIG. 4, a wand 26 of known length may be removeably secured to the instrument 12 to ensure images captured subsequent to the standard image are taken at the same distance from the wound area 16 as the standard image, and therefore, the size of those subsequent images may be reliably calculated. Alternatively, a simple disposable ruler can be placed in the field adjacent to the wound during photography. As another alternative, a binocular instrument 12 may be used to calculate the distance from the wound area and thereafter the relative size of the wound. Further, a laser range finder may be incorporated into the instrument. In one embodiment, the wand 26 has a tip 28 of a known size and/or with a ruler attached thereto to provide an alternative means for approximating wound size.

In another embodiment, the wand 26 may have a padded tip 28 to provide greater comfort when rested against a surface of a subject's skin adjacent a wound area. In another embodiment, the size of a wound area may necessitate resting the wand tip 28 within the wound area itself In this embodiment, it is envisioned that the wand 26 may have a removable, disposable sterile tip 28 that may be coated with an analgesic compound or other medicament (for example, an antibiotic) to reduce and/or avoid pain in the wound area upon use of the instrument to catalogue the wound area. The removable, disposable sterile tips 28 may be placed onto a wand 26 and then disposed of after use, whereas the wand is retained. Alternatively, the entire wand 26 may be provided as a removable, sterile item where the wand tip 28 is integral with the wand and may also be coated with an analgesic compound or other medicament (for example, an antibiotic).

In one embodiment, a plurality of disposable sterile wands 26 may be provided in a resealable package. In another embodiment, a plurality of removable, disposable sterile wand tips 28 may be provided in a resealable package. In another embodiment, a kit may include an instrument 12, one or more wands 26, one or more wand tips 28, and instructions for capturing image data. For example, the instructions may direct a user to attach a wand 26 to the instrument 12 and take a first calibration image with the wand touching a test surface to calibrate the instrument. The instructions may then direct the user to capture a first test image with the instrument 12 at one angle relative to the test surface and then direct the user to capture a second test image with the instrument at a second angle relative to the test surface. The instructions may then direct the user to send the test images to an interface 20. The instructions may then direct the user to replace the wand 26 or wand tip 28.

It is further envisioned that the configuration of the wand 26 as a single point contact device (for example, a device that stabilizes the instrument with respect to a surface by making contact with the surface at a single point) enables a user to more readily place and manipulate the relative position of the instrument 12 with respect to the wound area in a reproducible manner. For example, the instrument 12 may be pivoted while holding the wand tip 28 in place on the surface of the subject's skin, such that the wand moves like a joy stick to change the relative angle of the instrument with respect to the wound area. Image capture during manipulation of the instrument 12 while the wand tip 28 is placed against the surface of the subject's skin may enable creation of a three dimensional image of the surface. Alternatives to the wand configuration include bipod and tripod configurations and the like (not shown).

The size of images 14 captured may be displayed with a scale 30 along the X and Y axes of the image. In this way, a user may not only easily catalogue wound size at resident intake, but may also easily monitor changes in wound size (a metric for healing or wound growth) over time by taking subsequent images. The current standard of care practiced by wound care physicians and nurses includes obtaining wound measurements using disposable rulers. Wound appearance and condition are actually more critical than wound size at the time of intake. Following wound size over time, however, is the most effective measure of response to treatment and confirmation of wound healing. Wound healing speaks not only to the appropriateness of treatment but also to the resident's ability to heal.

The image data are compiled within the instrument 12 and/or processing unit 18 by a software program to provide a single or multiple images that enable clinically relevant consideration. In addition, these cumulative data may be automatically combined in the processing unit 18 into a modified Braden Risk Scale score or a modified range on the Braden Risk Scale obtained clinically. A Braden Risk Scale score of 15-16 is considered low risk for developing a wound, a score of 13-14 is considered moderate risk for developing a wound, a score of 12 or less is considered high risk. The current standard of care requires that Braden Scores be obtained at the time of admission, quarterly, after a major change and after return from a hospital. Many facilities initiate wound prevention protocols when the Braden Risk Score is less than 16. The maximum score on the Braden scale is 20. When a wound is present the processing unit 18 subtracts 5 from the Braden score entered by the clinical staff person performing the assessment and generates a modified Braden Risk Scale score. The presence of a wound increases the risk of further development of other wounds. The processing unit 18 generated modified Braden Risk Scale score alerts treating personnel to initiate higher risk protocols than the current Braden Scale score currently does.

Alternatively, the processing unit 18 may generate a modified Braden Risk Scale score automatically without initial input by clinical staff based on wound image data criteria including the type of wound, the size of the wound, the temperature of the wound, wound topography, the condition of the wound area, the location of the wound on the resident's body, and other clinical factors.

In another embodiment, the system 10 may be implemented, at least in part, in the form of an application or “App” in combination with one or more of a “smartphone,” tablet, and the like. In another embodiment, the system 10 may incorporate some or all aspects of those and variations of those described in U.S. Patent Application Publication No. 2013/0085777.

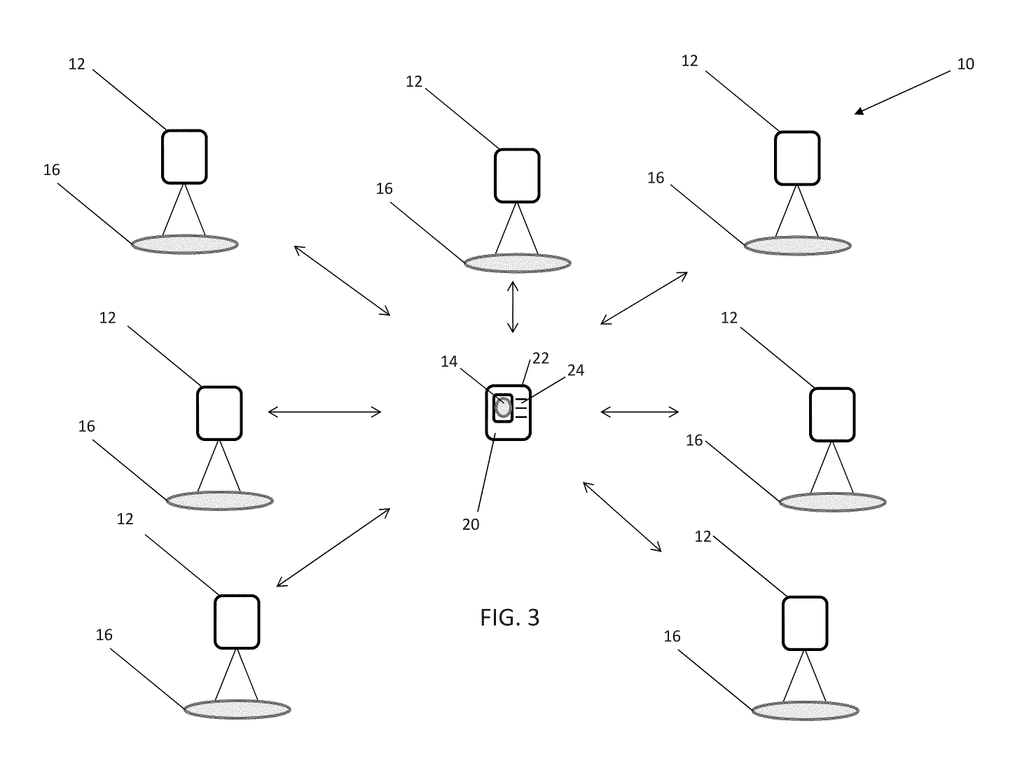

A particular advantage illustrated in FIG. 3 is the ability of the system 10 to incorporate multiple instruments 12 at different or the same nursing home to convey information remotely to a single interface 20. In this way, a single remote physician or other health care worker can administer wound diagnosis, a wound treatment regimen, a wound acknowledgment, or convey other information and/or other instructions, and the like for multiple residents in different rooms and/or at multiple facilities without the need to physically travel to each resident. Similarly, as seen in FIG. 4, a single instrument 12 can convey information to a multitude of interfaces 20 to enable multiple physicians and/or health care workers to consider the wounds and additional information of a single resident. In this way, a single resident can be diagnosed and taken care of by a plurality of individuals located at different places at the same time. According to CMS, advanced wound care in long term care facilities includes a multi-disciplinary team. Instrument 12 facilitates communication among team members.

Unless defined otherwise in this specification, technical and scientific terms used herein have the same meaning as commonly understood by one of ordinary skill in the art to which this invention belongs and by reference to published texts.

### INDUSTRIAL APPLICABILITY

The systems, methods, and system components disclosed herein provide better tools for long term care facilities, such as nursing homes, to improve nursing home resident wound care prevention.

Numerous modifications will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications which come within the scope of the application are reserved. All patents and publications are incorporated by reference.

What is claimed is:

1. A system for capturing an image of a wound area of a subject, comprising:
a. an instrument adapted to capture image data of a wound area of a subject;
b. a wand attached to the instrument; and
c. a wand tip adapted to be placed against at least one of a surface of the subject's skin adjacent the wound area and a surface of the wound area.

2. The system of claim 1, wherein the instrument comprises a camera.

3. The system of claim 2, wherein the wand is removably secured to the camera.

4. The system of claim 1, wherein the wand tip comprises an analgesic compound or other medicament.

5. The system of claim 4, wherein the wand and wand tip are integral.

6. The system of claim 4, wherein the wand tip is removable from the wand.

7. The system of claim 1, wherein the wand comprises a single point contact device adapted to enable a user to manipulate the relative position of the instrument with respect to the wound area in a repeatable manner without moving the wand tip.

8. A kit, comprising the system of claim 1.

9. The kit of claim 8 further comprising a resealable package comprising a plurality of removable, disposable sterile wand tips.

10. The kit of claim 8 further comprising instructions for capturing image data.

11. A system for providing wound information to a remote location, comprising:
a. an instrument adapted to capture image data of a wound area of a subject;
b. a processing unit associated with the instrument adapted to store, process, and/or transfer the image data to a remote location; and
c. an interface adapted to receive the image data and display an image comprising the image data,
wherein the image provides information on the interface to enable a user to clinically assess the wound area of the subject based on wound area appearance, wound area size, wound area condition, wound area topography, and wound type.

12. The system of claim 11, wherein the image comprises image data comprising two or more of wound area appearance, wound area size, wound area temperature, and wound area topography.

13. The system of claim 11, wherein the image provided on the interface comprises a scale along X and Y axes of the image.

14. The system of claim 11 further comprising an image standard for distance calibration of the instrument.

15. The system of claim 14, wherein the processing unit is adapted to calculate wound area size of the image based on the distance calibration of the instrument.

16. A method of providing wound information to a remote location, comprising:
a. capturing an image of a wound area of a subject using an instrument, wherein the image provides information on a remote interface to enable a remote user to clinically assess the wound area of the subject based on at least wound area appearance, wound area size, wound area condition, wound area topography, and wound type;
b. storing the image in a processing unit;
c. transferring the image to a remote location;
d. displaying the image on the remote interface; and
e. recording an acknowledgment of the remote user based on the wound appearance,
wherein the acknowledgment is transferred to the instrument and stored in the processing unit in association with the image.

17. The method of claim 16, wherein the image comprises image data comprising two or more of wound area appearance, wound area size, wound area temperature, and wound area topography.

18. The method of claim 17, wherein the image provided on the interface comprises a scale along X and Y axes of the image.

19. The method of claim 18, wherein the instrument comprises a removable wand for establishing a distance calibration of the instrument based on the length of the wand.

20. The method of claim 19, wherein the processing unit is adapted to calculate wound area size of the image based on the distance calibration of the instrument.
